(19) United States
(12) Patent Application Publication
(10) Pub. No.: US 2020/0316399 A1
(43) Pub. Date: Oct. 8, 2020

(54) Acupuncture Pen with Pulsed Electromagnetic Field Therapy (PEFT) Function

(71) Applicant: LIGHT TREE, Amstelveen (NL)

(72) Inventors: Alain Dijkstra, Amstelveen (NL); YVONNE JOHANNA MARGARETHA HOUTHUIJS, Amstelveen (NL)

(73) Assignee: LIGHT TREE, Amstelveen (NL)

(21) Appl. No.: 16/377,236

(22) Filed: Apr. 7, 2019

(51) Int. Cl. A61N 5/06; A61H 39/00

(52) U.S. Cl. CPC A61N 5/0619; A61N 5/0616; A61H 39/00; A61N 2005/0644; A61H 2039/005; A61N 2005/0651; A61N 2005/0659; A61N 2005/0662; A61N 2005/0626

(57) ABSTRACT

The invention is a device, system, and method of implementing pulsed electromagnetic field (PEMF) therapy, light therapy and acupuncture therapy in a handheld device that can be operated by a user without expert supervision. The present invention comprises of acupuncture pen with pulsed electromagnetic field therapy (PEMFT) function and light therapy. The acupuncture pen includes a detachably attached pointed member to operate when in perfect contact with skin, a pulsed electromagnetic field (PEMF) unit, Light emitting unit, a power source and circuit controller connected to each other through electrical communication. The acupuncture pen also helps a user by pointing to next acupuncture point with laser guiding means.

### FIELD OF THE INVENTION

The present invention relates to an Integrating Pulsed Electromagnetic Field (PEMF) therapy within acupuncture pen for enhancing body's overall functioning, e.g. skin rejuvenation, blood circulation, body pain etc. by sending energy waves towards affected/pained areas of the body.

### DESCRIPTION OF THE BACKGROUND ART

According to medical science, every cell in the human body is essentially a battery, carrying an electrical charge. Disruption of electromagnetic energy in cells causes impaired cell metabolism and is a cause for health disorders such as for chronic diseases etc. Cellular impairment hampers the human body's rejuvenation and affects especially during acute injury cases. Pulsed Electromagnetic Field Therapy (PEMFT) helps to recharge cells and aids in the body's natural healing process. PEMF therapy leads to improved function by allowing an increased expulsion of toxins, as well as better nutrient and oxygen flow into the cells. The PEMF radiations passes through cells completely without affecting cells, and increase the spin of the electrons. As a result, wellness levels are improved and the body functions work more efficiently.

Further, the PEMF therapy aids in the body's recovery process by supporting healthy joint and cartilage function, as well as supporting healthy bone structure. By creating better functioning, healthier cells, PEMF therapy is able to provide support for stimulated tissue healing, healthy bone structure, increased immune system support, reduced inflammation, stress reduction, enhanced production of calming neurotransmitters, decreased nervous irritability, increased relaxation, deeper & more restorative sleep, increased expulsion of toxins, improved nutrient absorption, reduced post-exercise pain and reduced absent-mindedness. In the prior art, the electromagnetic field therapy is being used by the user for skin treatment or for other body function treatment as mentioned below.

In the U.S. Pat. No. 8,412,328B2 Andrew J. Whelan et.al discloses an electromagnetic therapy device for applying an electromagnetic field to bodily tissue include a self-contained and portable electromagnetic field generating device disposed over a surface of body tissue such that the radiated electromagnetic fields impinge upon the body tissue. However, the prior art not providing acupuncture and light therapy treatment in a single device and the device has a complex structure and a plurality of elements thereby the device is not affordable for an individual user.

In an another US patent application No. 2016,015,856,8A1 Janez Uplaznik et.al discloses a handheld combined magnetic and light therapy device having a treatment surface that is to be positioned adjacent to the body part to be treated during operation. But the device is not providing acupuncture treatment and further device have multiple hardware components adding extra manufacturing cost.

The above-mentioned references are mentioned herein to understand the present invention. Further, the above-mentioned references fail to disclose a device/technique that can implement an acupuncture pen with PEMF. There is a need for a device that can operate as an acupuncture instrument without help from a practitioner. Also, a device that can provide PEMF as well as light therapy to the user.

### BRIEF SUMMARY OF THE INVENTION

In order to overcome the disadvantages of the prior art, the present invention provides a device, system, and method for providing light therapy along with a handheld acupuncture device that can be operated without the help of any specialist or expert.

In one aspect of the present invention, an acupuncture pen with pulsed electromagnetic field therapy (PEMFT) function is disclosed. The acupuncture pen comprises of a housing including a detachably attached pointed member to operate when comes in contact with a user skin, a pulsed electromagnetic field (PEMF) unit, light emitting unit, a power source and a circuit controller connected to each other through electrical communication. The existing devices have no means by which a user can gets to know about the contact of the pointer member on his/her skin, to overcome the problem, in the present device the circuit controller is adapted to receive a notification of the proper contact with skin via the detachably attached pointed member, and is adapted to activate the PEMF unit and light-emitting unit to emit pulsed electromagnetic field (PEMF) and light therapy to the contacted skin via a detachably attached pointed member along with acupuncture therapy.

In another aspect of the present invention, a system for implementing Pulsed Electromagnetic Field Therapy (PEMFT) using an acupuncture pen is disclosed. The system comprising of an acupuncture pen with Pulsed Electromagnetic Field Therapy (PEFT) function, wherein a housing including a detachably attached pointed member to operate when in a proper contact with the skin, a Pulsed Electromagnetic Field (PEMF) unit, Light emitting unit, a power source and circuit controller connected to each other through electrical communication. The circuit controller is adapted to receive a notification of the perfect contact with skin via the detachably attached pointed member, and is adapted to activate the (PEMF) unit and Light-emitting unit which emits Pulsed Electromagnetic Field (PEMF) and light therapy to the contacted skin via a detachably attached pointed member along with acupuncture therapy. The system further comprising an electronic device connected to the acupuncture pen through a network and a transceiver of the circuit controller.

In yet another aspect of the present invention, a method of implementing Pulsed Electromagnetic Field Therapy (PEMFT) using an acupuncture pen is disclosed. The method comprises of placing the acupuncture pen on skin of a user, wherein a detachably attached pointed member of the acupuncture pen emits pulsed electromagnetic field (PEMF) and light therapy when in a proper contact with the skin and a notification means of the acupuncture pen notifies the user using a light emitting diode (LED) when the acupuncture pen is active.

The present invention has been made in view of a device implementing at least one of pulsed electromagnetic therapy, light therapy and acupuncture therapy within acupuncture pen. Further, the acupuncture device is portable and implements PEMF and light therapy therein, the device helps to increase production of nitric oxide, improves microcirculation, increases oxygen, ions, and nutrients in cells. The device further increases oxygen partial pressure, increases ATP production by electrical stimulation, stimulates RNA and DNA production. The device accelerates protein biosynthesis by electron and energy transfer, regulates ant oxidative by increased circulation of available electrons, calcium transport to strengthen bones, joints, and muscles. Increased absorption, increased tissue elasticity of the cells with increased collagen production, increased cellular genes that promote bone and cartilage tendons and soft tissue growth, stimulate cellular repair mechanisms etc., are also advantages of the acupuncture device.

According to the aspect of the any aspect of the present disclosure, the device is a single unit and is handheld, unlike the existing devices. Further, the device can use different shapes of the detachable pointed member.

The foregoing has outlined rather broadly the features and technical advantages of the present invention in order that the detailed description of the invention that follows may be better understood. Additional features and advantages of embodiments of the invention will be described hereinafter which form the subject claims of the invention. The conception and specific embodiment disclosed herein may be readily utilized as a basis for modifying or designing other structures for carrying out the same or similar purposes of the present invention. Please note that such equivalent constructions do not depart from the scope of the invention as set forth in the appended claims. The novel features which are characteristics of the invention, both as to its organization and method of operation together with further objects and advantages, which will be better understood from the following description when considered in connection with the accompanying figures. Each of the figures is provided for the purpose of illustration and description only and is not intended as a limitation of the scope of the present invention or appended claims.

### BRIEF DESCRIPTION OF THE DRAWINGS

FIG. 1: Illustrates a block diagram of an acupuncture pen with PEMF according to one embodiment of the present invention;

FIGS. 2 and 2A: Illustrates a plurality of views of the acupuncture device with PEMF according to one embodiment of the present invention;

FIG. 3: Illustrates a system for implementing pulsed electromagnetic field therapy (PEMFT) using an acupuncture pen according to another embodiment of the present invention; and

FIGS. 4 and 4A: Illustrates an acupuncture pen with PEMF and wireless charging units according to the various embodiment described herein.

### DETAILED DESCRIPTION OF THE DISCLOSURE

Embodiments of the present disclosure will be described more fully hereinafter with reference to the accompanying drawings in which like numerals represent like elements throughout the figures, and in which example embodiments are shown. Embodiments of the claims may, however, be embodied in many different forms and should not be construed as limited to the embodiments set forth herein.

Certain terminology used in the following description is for illustration only and is not limiting to the invention. The words “right,” “left,” “lower” “upper”, “Back” and “front” designate directions in the drawings to which reference is made. The terminology includes the words which are specifically mentioned above, derivatives thereof and words of similar import.

The present invention has been made in view of a device implementing at least one of pulsed electromagnetic therapy, light therapy and acupuncture therapy within acupuncture pen. The acupuncture pen includes a detachably attached pointed member to operate when comes in contact with a user skin, a Pulsed Electromagnetic Field (PEMF) unit, Light emitting unit, a power source and circuit controller connected to each other through electrical communication. The acupuncture pen also helps a user by pointing to next acupuncture point with laser guiding means.

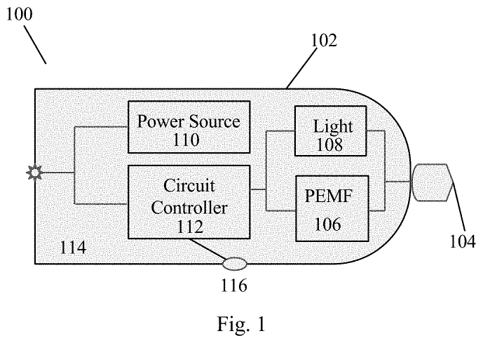

FIG. 1 illustrates acupuncture pen 100 in accordance with one embodiment of the present invention. The acupuncture pen 100 comprises of a housing 102, a detachably attached pointed member 104, a Pulsed Electromagnetic Field (PEMF) unit 106, a light emitting unit or a LED unit 108, a power source 110 and a circuit controller 112. The components of the acupuncture pen 100 are coupled electrically within the housing 102. The acupuncture pen also includes a switch 116 to turn on power for PEMF unit 106 and light emitting unit 108, wherein the switch 116 is a toggle or push switch. The acupuncture pen 100 further comprises of notification means, such as but not limited to LED unit 114, an audio speaker and haptic feedback means.

The acupuncture pen 100 can provide but not limited to PEMF therapy, light therapy and acupuncture therapy, wherein the detachably attached pointed member 104 provides at least one of the therapies to a user when in proper contact with skin. In another embodiment, the detachably attached pointed member 104 also includes a touch sensor to identify the proper contact with the skin.

Still with reference to FIG. 1, the circuit controller 112 is adapted to receive a notification of the proper contact with skin via the detachably attached pointed member 104 and is adapted to activate the PEMF unit 106 and light emitting unit 108 for emitting PEMF and light therapy to the contacted skin via a detachably attached pointed member along with acupuncture therapy. The circuit controller 112 comprises of electronic chip, memory, and transceiver, wherein the electronic chip processes command stored in the memory, and the acupuncture pen connects to a network using the transceiver. Further, the notification means connected to the power source 110 and the circuit controller 112 is used to notify the user when comes in proper contact with the user skin. In an embodiment, a second pointed member with different intensity of, PEMF and light therapy, and/or a different surface area than the detachably attached pointed member 104 is connected to the housing 102 of the acupuncture pen 100, allowing versatile application of the acupuncture pen 100.

In an exemplary embodiment of the present invention, the light emitting unit 108 gives light therapy to the user, the light therapy comprises of visible and/or infrared (IR) light.

The acupuncture pen 100 further comprising a laser guiding unit attached to the circuit controller 112 to project laser at next acupuncture point on the skin, wherein the circuit controller 112 receives the next acupuncture point from the network through the transceiver.

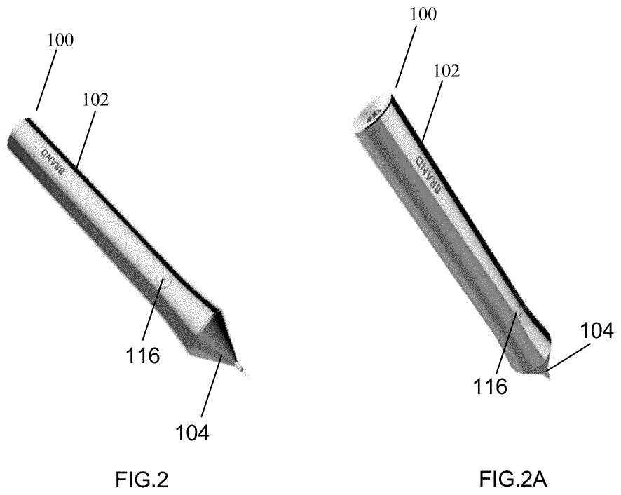

Referring to FIGS. 2 and 2a, are illustrating different views of the acupuncture pen 100 with the housing 102, switch 116, detachably attached pointed member 104 and LED unit 114.

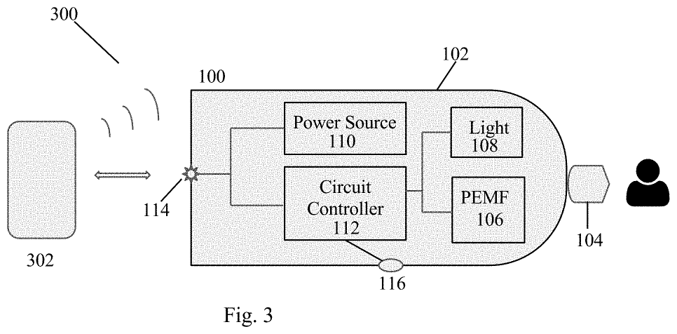

Referring to FIG. 3 according to another embodiment of the present disclosure a system 300 involving the acupuncture pen 100 connected to an electronic device 302, wherein, the electronic device 302 communicates with the acupuncture pen 100 about next acupuncture point. The electronic device 302 displays a virtual human body to point the next acupuncture point and/or scans the user using camera to point the next acupuncture point. In another embodiment, the electronic device 302 transmits the next acupuncture point to the acupuncture pen 100 through the network. Wherein the electronic device is not limited to a mobile phone, laptop, and tablet.

In yet another embodiment, the electronic device 302 stores a user profile during operation of the acupuncture device 100 and performs machine learning to provide feedback for future use/operation. The electronic device 302 also shares new acupuncture points with the acupuncture pen 100 to project through laser guiding means.

In another embodiment, the present invention is a method of implementing pulsed electromagnetic field (PEMF) therapy using an acupuncture pen. The method includes placing the acupuncture pen 100 on the skin of a user and selecting an operation mode of the acupuncture pen, wherein operation mode includes PEMF therapy, light therapy, and acupuncture therapy. The method also includes selecting operation mode using switch 116. The method then emits pulsed electromagnetic field (PEMF) and light therapy when in proper contact with the skin through a detachably attached pointed member 104 of the acupuncture pen 100. The method further includes notifying the user using a light emitting diode (LED) when the acupuncture pen 100 is active.

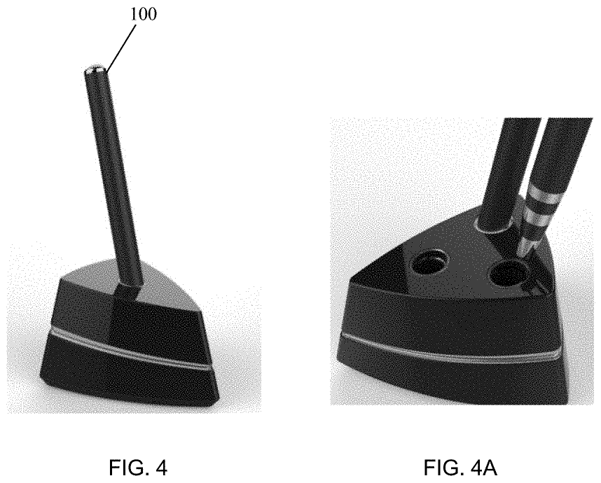

Referring to FIGS. 4 and 4a, the acupuncture pen 100 supports wireless charging according to any of the embodiment described herein.

As disclosed by various embodiments of the present invention, the device, system, and method of implementing Pulsed Electromagnetic Field Therapy (PEMFT) using an acupuncture pen which helps a user to operate it without the help of an expert. Further, the device can be operated as PEMF therapy, light therapy and acupuncture therapy simultaneously or individually. The device can be further connected to an electronic device and can be guided by lasers.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Although specific embodiments and certain structural arrangements have been illustrated and described herein, it will be clear to those skilled in the art that various other modifications and embodiments may be made incorporating the spirit and scope of the underlying inventive concepts and that the same is not limited to the particular methods and structure herein shown and described except in so far as determined by the scope of the appended claims.

### LIST OF COMPONENTS

100: acupuncture pen

102: Housing

104: Pointed Member

106: PEMF Unit

108: LED Unit

110: Power Source

112: Circuit Controller

114: LED Unit

116: Switch

300: System

302: Electronics Device

What is claimed is:

1. An acupuncture pen with pulsed electromagnetic field therapy (PEMFT) function, the acupuncture pen comprising:
a housing including a detachably attached pointed member to operate when in perfect contact with skin, a pulsed electromagnetic field (PEMF) unit, a light emitting unit, a power source and circuit controller connected each other through electrical communication, wherein:
the circuit controller is adapted to receive a notification of the perfect contact with skin via the detachably attached pointed member,
the circuit controller is adapted to activate the PEMF unit and the light-emitting unit to emit a pulsed electromagnetic field (PEMF) and light therapy to the contacted skin via a detachably attached pointed member along with acupuncture therapy.

2. The acupuncture pen according to claim 1, wherein the circuit controller further comprising, an electronic chip, memory, and transceiver, wherein the electronic chip processes command stored in the memory, and the acupuncture pen connects to a network using the transceiver.

3. The acupuncture pen according to claim 1, a notification means connected to the power source and the circuit controller to notify a user of proper contact of the attached pointed member with the skin, the notification means comprising at least one of LED unit, an audio speaker and haptic feedback means.

4. The acupuncture pen according to claim 1, wherein the detachably attached pointed member includes a touch sensor to identify the perfect contact with skin.

5. The acupuncture pen according to claim 1, wherein the light emitting unit gives light therapy to a user, the light therapy comprises of visible and/or infrared (IR) light.

6. The acupuncture pen according to claim 1, wherein a second pointed member with different intensity of, PEMF and light therapy, and/or a different surface area than the detachably attached pointed member is connected to the housing of the acupuncture pen.

7. The acupuncture pen according to claim 1 further comprising a laser guiding unit attached to the circuit controller to project laser at next acupuncture point on the skin, wherein the circuit controller receives a next acupuncture point from a network through a transceiver.

8. A system for implementing pulsed electromagnetic field therapy (PEMFT) using an acupuncture pen comprising:
the acupuncture pen with pulsed electromagnetic field therapy (PEFT) function, the acupuncture pen comprising:
a housing including a detachably attached pointed member to operate when in perfect contact with skin, a pulsed electromagnetic field (PEMF) unit, Light emitting unit, a power source and circuit controller connected to each other through electrical communication, wherein:
the circuit controller is adapted to receive a notification of the perfect contact with skin via the detachably attached pointed member,
the circuit controller is adapted to activate the PEMF unit and Light-emitting unit to emit pulsed electromagnetic field (PEMF) and light therapy to the contacted skin via a detachably attached pointed member along with the acupuncture therapy; and
an electronic device connected to the acupuncture pen through a network and a transceiver of the circuit controller.

9. The system according to claim 8, wherein the circuit controller further comprising an electronic chip, memory, and the transceiver, wherein the electronic chip processes command stored in the memory, and the acupuncture pen connects to a network using the transceiver.

10. The system according to claim 8, a notification means connected to the power source and the circuit controller of the acupuncture pen to notify a user of a perfect contact with the skin, the notification means comprising at least one of LED unit, an audio speaker and haptic feedback means.

11. The system according to claim 8, wherein the detachably attached pointed member includes a touch sensor to identify the perfect contact with the skin.

12. The system according to claim 8, wherein the light emitting unit gives light therapy to a user, the light therapy comprises of visible and/or infrared (IR) light.

13. The system according to claim 8, wherein a second pointed member with different intensity of, PEMF and light therapy, and/or a different surface area than the detachably attached pointed member is connected to the housing of the acupuncture pen.

14. The system according to claim 8, the electronic device communicates with the acupuncture pen about next acupuncture point, wherein the electronic device displays a virtual human body to point the next acupuncture point and/or scans a user using camera to point the next acupuncture point.

15. The system according to claim 8 further comprising a laser guiding unit attached to the circuit controller to project laser at next acupuncture point on the skin, wherein the circuit controller receives a next acupuncture point from a network and/or the electronic device through the transceiver.

16. A method of implementing Pulsed Electromagnetic Field Therapy (PEMFT) using an acupuncture pen comprises of:
placing the acupuncture pen on skin of a user, wherein a detachably attached pointed member of the acupuncture pen emits pulsed electromagnetic field (PEMF) and light therapy when placed in a proper contact with the skin and a notification means of the acupuncture pen notifies the user using a light emitting diode (LED) when the acupuncture pen is active.
